SEQUENCE LISTING (12 sequences)

SEQ ID NO: 1 - DNA, 20 bp, Artificial Sequence
  PCR primer 167
gctggtccgagaggatgatc

SEQ ID NO: 2 - DNA, 20 bp, Artificial Sequence
  PCR primer 166
gttatagttacggccgccgt

SEQ ID NO: 3 - DNA, 5373 bp, Lemna minor
  misc_feature 3530, 4789: n = A,T,C or G
gctggtccgagaggatgatcagccacactgggactgagacacggcccagactcctacgggaggcagcagtggggaattttccgcaatgggcgaaagcctgacggagcaatgccgcgtggaggtagaaggcccacgggtcgtgaacttcttttctcggagaagaagcaatgacggtatctgaggaataagcatcggctaactctgtgccagcagccgcggtaagacagaggatgcaagcgttatccggaatgattgggcgtaaagcgtctgtaggtggcttttcaagtccgtcgtcaaatcccagggctcaaccctggacaggcggtggaaactaccaagctggagtacggtaggggcagagggaatttccggtggagcggtgaaatgcgtagagatcggaaagaacaccaatggcgaaagcactctgctgggccgacactgacactgagagacgaaagctaggggagcgaatgggattagataccccagtagtcctagccgtaaacgatggatactaggcgctgtgcgtatcgacccgtgcagtgctgtagctaacgcgttaagtatcccgcctggggagtacgttcgcaagaatgaaactcaaaggaattgacgggggcccgcacaagcggtggagcatgtggtttaattcgatgcaaagcgaagaaccttaccagggcttgacatgccgcgaatcctcttgaaagagaggggtgccttcgggaacgcggacacaggtggtgcatggctgtcgtcagctcgtgccgtaaggtgttgggttaagtcccgcaacgagcgcaaccctcgtgtttagttgccaccattgtggaaccctgaacagactgccggtgataagccggaggaaggtgaggatgacgtcaagtcatcatgccccttatgccctgggcgacacacgtgctacaatggccgggacaaagggtcgcgatcccgcgagggtgagctaaccccaaaaacccgtcctcagttcggattgcaggctgcaactcgcctgcatgaagccggaatcgctagtaatcgccggtcagccatacggcggtgaattcgttcccgggccttgtacacaccgcccgtcacactatgggagctggccatgcccgaagtcgttaccttaaccgcaaggggggggatgccgaaggcggggctagtgactggagtgaagtcgtaacaaggtagccgtactggaaggtgcggctggatcacctccttttcagggagagctaatgcttgttgagtattttttttggtttgacactgcttcacacccaaaaagaagcgagctacgtctgagctaagcttggatatggaagtcttctttcgtttcttgacggtgaaataagaccaagctcatgagcttattatcctaggtcggaacaagttgataggatccttttacgtccccatgtccctcccgtgtggacacatggggacgtaaaaaggaaagagagggatggggtttctctcgcttttggcatagcaggcctcccggtgggaggcccgcacgacgggctattagctcagtggtagagcgcgcccctgataattgcgtcgttgtgcctgggctgtgagggctctcagccacatggatagttcaatgtgctcatcagcgcctgacccggagatgtggatcatccaaggcacattagcatggcgtactcctcctgttcgaatcggagtttgaaaccaaacttatcctcaggaggatagatggggcgattcaggtgagatccaatgtagatccaactttctattcactcgtgggatccgggcggtccgggggggaccaacaaggctcctctcttctcgagaatccatacatcccttatcagtgtatggacagctatctctcgagcacaggtttaggttcggcctcaacgggaaaatggagcacctaacaacgcatcttcacagaccaagaactacgagatcaccccttttattctggggtgacggagggatcgtaccattcgagccttttttcatgcttttcctgaaggtctggagaaacgtgaaattctttttatctatctcttgactcgaaatgggagcaggtttgaaaaaggatcttagagtgtctagggttgggccaggagggtttcttaacctcttcttttttcttcccatcggagttttttcacaaagacttccatggtaagggggaagggtggaacaagcaaagcacacttgtagagcgcagtacagcggagagctgtatgctgcgttcgggaaggatgaatcgctcctgaaaaagaatctattgattctcccccaattgtttggatcgtaggtgcgatgatttacttcacgggcgaggtctctggttcaagtccaggatggcccagcggcgccagggaaaagaatagaagaagcatctgactccttcatgcatgctccacttggctcggggggatacagctcagttggtagagctccgctcttgcaattgggtcgttgcgattacgggttggatgtctaattgtccaggcggtaatgatagtatcttgtacctgaactggtggctcactttttctaagtaatggggaagaggaccgaaacatgccactgaaagactctactgagacaaagatgggctgtcaagaacgtagaggaggtaggatgggcagttggtcagatctagtatggatcgtatatggaccttagttggagtcggcggctctcctagggttccctcatctgggatccctggggaagaggatcaagttggcacttgcgaacagcttgatgcactatctcccttcaaccctttgcgcgaaatgtgacaaaaggaaggaaaatccatggaccgaccccatcgtctccaccccgtaggaactacgagatcaccccagggacgccttcggcatccaggggtcacggaccgaccatagatcctgttcaataagtggaacgcattagcagtccgttctctggttgggcagtaagggtcggagaagggcaatcactcgttcttaaaaccagcattcttaaaacccaagagtcgggcagaaaaagggggagagttctccgttcctgattctcctgtagctggattctccggaaccacaagaatccttagaatgggattccgactcagcaccttttgatattttgagaagagttgctctttggagagcacagtacgatgaaagttgtaagctgtgttcgggggggagttattgtctatcgttggcctctatagtagaatcagtcggggaggcccgagaggcggtggtttaccctgtggcggatgtcagcggttcgagtccgcttatctccagcccgtgaacttagccgaaactatgatagcacccaattttgacgattcggcagttcgatctatgatttatcattcatggacgttgataagatccttccatttagcagcaccttaggatggcatagccttaacatggcgaggttcaaacgaggaaaggcttacggtggatacctaggcacccagagacgaggaagggcgtagtaagcgacgaaatgcttcggggagttgaaaatangcatagatccggagattcccgaataggttaacctttcgaactgctgctgaatccatgggcaggcaagagacaacctggcgaactgaaacatcttagtagccagaggaaaagaaagcaaaagcgattcccgtagtagcggcgagcgaaatgggagcagcctaaaccgtgaaaacggggttgtgggagagcaatacaagcgtcgtgctgctaggcgaagcggttgaatactgcaccctagatggcgaaagtccagtagccgaaagcatcactagcttacgctctgacccgagtagcatggggcacgtggaatcccgtgtgaatcagcaaggaccaccttgcaaggctaaatactcctgggtgaccgatagcgaagtagtaccgtgagggaaaggtgaaaagaacccccatcggggagtgaaatagaacatgaaaccgtgagctctcaagcagtgggaggagaatctgatctctgaccgtgtgcctgttgaagaatgagccggcgactcataggcagtggcttggttaagggaatccaccggagccgtagcgaaagcgagtcttcatagggcgattgtcactgcttatggacccgaacctgggtgatctatccatgaccaggatgaagcttgggtgaaactaagtggaggtccgaaccgactgatgttgaagaatcagcggatgagttgtggttaggggtgaaatgccactcgaacccagagctagctggttctccccgaaatgcgttgaggcgcagcagttgactggacatctaggggtaaagcactgtttcggtgcgggccgcgagagcggtaccaaatcgaggcaaactctgaatactagatatgaccccaaaataacggaggtcaaggtcggccagtgagacgatgggggataagcttcatcgtcgagagggaaacagcccagatcaccagctaaggcccctaaatgaccgctcagtgataaaggaggtaggagtgcagagacagccaggaggtttgcctagaagcagccacccttgaaagagtgcgtaatagctcactgatcgagcgctcttgcgccgaagatgaacggggctaagcgatctgccgaagctgtgggatgtaaaaatgcatcggtaggggagcgttccgccttagatagaagcacccgtgcaagcaggtgtggacgaagcggaagcgaaaatgtcggcttgagtaacgcaaacattggtgagaatccaaatgccccgaaaacctaagggttcctccgcaaggttcgtnccacggagggtgagtcagggcctaagatcaggccgaaaggcgtagtcgatggacaacaggtgaatattcctgtactaccccttgttggtcccgagggacggaggaggctaggttagccgaaagatggttatcggttcaaggacgcaaggtgaccctgatttttcagggtaagaaggggtagagaaaatgcctcgagccaatgtctgagtaccaggcgctacggcgctgaagtaacccatgccatactcccaggaaaagctcgaacgaccttaaacaagagggtacctgtacccgaaaccgacacaggtgggtaggtagagaatacctaggggcgcgagacaactctctctaaggaactcggcaaaatagccccgtaacttcgggagaaggggtgcctcctcacaaagggggtcgcagtgaccaggcccgggcgactgtttaccaaaaacacaggtctccgcaaagtcgtaagaccatgtatgggggctgacgcctgcccagtgccggaaggtcaaggaagttggtgacctgatgacaggggagccagcgaccgaagccccggtgaacggcggccgtaactaaac

SEQ ID NO: 4 - DNA, 30 bp, Artificial Sequence
  PCR Primer 207
acaaggtagccgtactggaaggtgcggctg

SEQ ID NO: 5 - DNA, 24 bp, Artificial Sequence
  PCR Primer 203
ttcaactccccgaagcatttcgtc

SEQ ID NO: 6 - DNA, 119 bp, Nicotiana tabacum
gctcccccgccgtcgttcaatgagaatggataagaggctcgtgggattgacgtgagggggcagggatggctatatttctgggagcgaactccgggcgaatacgaagcgcttggatacag

SEQ ID NO: 7 - DNA, 16 bp, Nicotiana tabacum
ttgtagggagggattt

SEQ ID NO: 8 - DNA, 189 bp, Nicotiana tabacaum
gatcctggcctagtctataggaggttttgaaaagaaaggagcaataatcattttcttgttctatcaagagggtgctattgctcctttctttttttctttttatttatttactagtattttacttacatagacttttttgtttacattatagaaaaagaaggagaggttattttcttgcatttattcatg

SEQ ID NO: 9 - DNA, 25 bp, Artificial Sequence
  PCR Primer 71
aaaacccgtcctcagttcggattgc

SEQ ID NO: 10 - DNA, 25 bp, Artificial Sequence
  PCR Primer 248
ccgcgttgtttcatcaagccttacg

SEQ ID NO: 11 - DNA, 24 bp, Artificial Sequence
  PCR Primer 210
ctgtagaagtcaccattgttgtgc

SEQ ID NO: 12 - DNA, 20 bp, Artificial Sequence
  PCR Primer 234
ggttcggacctccacttagt

(19) United States
(12) Patent Application Publication
(10) Pub. No.: US 2005/0044593 A1
(43) Pub. Date: Feb. 24, 2005

(54) Chloroplast transformation of duckweed

(75) Inventors: Kevin Cox, Raleigh, NC (US); Charles G. Peele, Apex, NC (US)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Biolex, Inc., Pittsboro, NC (US)

(21) Appl. No.: 10/881,813

(22) Filed: Jun. 30, 2004

Related U.S. Application Data
(60) Provisional application No. 60/484,166, filed on Jul. 1, 2003.
(60) Provisional application No. 60/492,179, filed on Aug. 1, 2003.

(51) Int. Cl. A01H 1/00; C12N 15/82; A01H 9/00

(52) U.S. Cl. 800/288; 800/295

(57) ABSTRACT

The present invention provides methods and compositions for the transformation of duckweed plastids. The compositions and methods of the invention are useful in increasing the recombinant protein production capacity of the duckweed expression system. The compositions of the invention include transformed duckweed plastids and transplastomic duckweed cells and plants, as well as nucleic acid constructs useful for transforming duckweed plastids. The invention also provides methods for introducing one or more heterologous nucleotide sequences into a duckweed plastome.

### CROSS-REFERENCE TO RELATED APPLICATIONS

This application claims the benefit of U.S. Provisional Application Ser. Nos. 60/484,166, filed Jul. 1, 2003, and 60/492,179, filed Aug. 1, 2003, each of which is hereby incorporated in its entirety by reference herein.

### FIELD OF THE INVENTION

The present invention relates to methods and compositions for the transformation of duckweed plastids.

### BACKGROUND OF THE INVENTION

The duckweeds are the sole members of the monocotyledonous family Lemnaceae. The five genera and 38 species are all small, free-floating, fresh-water plants whose geographical range spans the entire globe (Landolt (1986) Biosystematic Investigation on the Family of Duckweeds: The Family of Lemnaceae—A Monograph Study Geobatanischen Institut ETH, Stiftung Rubel, Zurich). Although the most morphologically reduced plants known, most duckweed species have all the tissues and organs of much larger plants, including roots, stems, flowers, seeds and fronds. Duckweed species have been studied extensively and a substantial literature exists detailing their ecology, systematics, life cycle, metabolism, disease and pest susceptibility, their reproductive biology, genetic structure, and cell biology. (Hillman (1961) Bot. Review 27: 221; Landolt (1986) Biosystematic Investigation on the Family of Duckweeds: The Family of Lemnaceae—A Monograph Study Geobatanischen Institut ETH, Stiftung Rubel, Zurich).

The growth habit of the duckweeds is ideal for microbial culturing methods. The plant rapidly proliferates through vegetative budding of new fronds, in a macroscopic manner analogous to asexual propagation in yeast. This proliferation occurs by vegetative budding from meristematic cells. The meristematic region is small and is found on the ventral surface of the frond. Meristematic cells lie in two pockets, one on each side of the frond midvein. The small midvein region is also the site from which the root originates and the stem arises that connects each frond to its mother frond. The meristematic pocket is protected by a tissue flap. Fronds bud alternately from these pockets. Doubling times vary by species and are as short as 20-24 hours (Landolt (1957) Ber. Schweiz. Bot. Ges. 67: 271; Chang et al. (1977) Bull. Inst. Chem. Acad. Sin. 24:19; Datko and Mudd (1970) Plant Physiol. 65:16; Venkataraman et al. (1970) Z. Pflanzenphysiol. 62: 316).

Intensive culture of duckweed results in the highest rates of biomass accumulation per unit time (Landolt and Kandeler (1987) The Family of Lemnaceae—A Monographic Study Vol. 2: Phytochemistry, Physiology, Application, Bibliography, Veroffentlichungen des Geobotanischen Institutes ETH, Stiftung Rubel, Zurich), with dry weight accumulation ranging from 6-15% of fresh weight (Tillberg et al. (1979) Physiol. Plant. 46:5; Landolt (1957) Ber. Schweiz. Bot. Ges. 67:271; Stomp, unpublished data). Protein content of a number of duckweed species grown under varying conditions has been reported to range from 15-45% dry weight (Chang et al (1977) Bull. Inst. Chem. Acad. Sin. 24:19; Chang and Chui (1978) Z. Pflanzenphysiol. 89:91; Porath et al. (1979) Aquatic Botany 7:272; Appenroth et al. (1982) Biochem. Physiol. Pflanz. 177:251). Using these values, the level of protein production per liter of medium in duckweed is on the same order of magnitude as yeast gene expression systems.

Accordingly, methods of expressing recombinant proteins in duckweed are useful for a number of research and commercial applications. For plant molecular biology research as a whole, a differentiated plant system that can be manipulated with the laboratory convenience of yeast provides a very fast system in which to analyze the developmental and physiological roles of isolated genes. For commercial production of valuable polypeptides, a duckweed-based system has a number of advantages over existing microbial or cell culture systems. Plants demonstrate post-translational processing that is similar to mammalian cells, overcoming one major problem associated with the microbial cell production of biologically active mammalian polypeptides, and it has been shown by others (Hiatt (1990) Nature 334:469) that plant systems have the ability to assemble multi-subunit proteins, an ability often lacking in microbial systems. Scale-up of duckweed biomass to levels necessary for commercial production of recombinant proteins is faster and more cost efficient than similar scale-up of mammalian cells, and unlike other suggested plant production systems, e.g., soybeans and tobacco, duckweed can be grown in fully contained and controlled biomass production vessels, making the system's integration into existing protein production industrial infrastructure far easier.

Methods of transforming the nuclear genome of duckweed have been described. See, for example, U.S. Pat. No. 6,040,498. Duckweed plant or duckweed nodule cultures can be efficiently transformed with an expression cassette containing a nucleotide sequence of interest by any one of a number of methods including Agrobacterium-mediated gene transfer, ballistic bombardment, or electroporation. However, the stable transformation of a duckweed plastid genome has not been described. Transformation of duckweed plastids would be advantageous because there are multiple copies of the plastome in each chloroplast and multiple chloroplasts in each cell and therefore high numbers of integrated transgenes can be obtained in each plant cell using plastome transformation methods.

The characteristics of the duckweed system make it an ideal choice to develop as an efficient, plant-based system for the production of recombinant proteins. Accordingly, the present invention provides methods and compositions for the transformation of duckweed plastids.

### SUMMARY OF THE INVENTION

The present invention provides duckweed plants having plastids that are transformed with one or more heterologous nucleotide sequences and nucleic acid constructs and methods useful for producing these transplastomic duckweed plants. Transformed duckweed plastids and duckweed cells containing these transformed plastids are also provided.

In some embodiments, the duckweed plant has plastids that are transformed with a heterologous nucleotide sequence encoding a polypeptide of interest. In further embodiments, more than one polypeptide of interest is encoded by the heterologous nucleotide sequence. The heterologous nucleotide sequence may contain a coding sequence for a maker polypeptide useful for the selection of transformed duckweed cells.

The plastids of the duckweed plant that are transformed with the heterologous nucleotide sequence may be proplastids, amyloplasts, chromoplasts, chloroplasts, etioplasts or leucoplasts.

The nucleic acid constructs of the invention have a first targeting nucleotide sequence that is homologous to a duckweed plastome sequence, at least one nucleotide sequence encoding one or more polypeptides of interest where the nucleotide sequence is heterologous to the duckweed plastome; and a second targeting sequence that is homologous to a duckweed plastome sequence. In some embodiments, the nucleic acid construct comprises an expression control sequence operably linked to the nucleotide sequence encoding the polypeptide of interest.

In some embodiments, the nucleic acid constructs for chloroplast transformation have a nucleotide sequence that increases the efficiency of the translation of the polypeptide of interest in duckweed. The nucleic acid construct may also contain one or more transcription termination sequences operably linked to the sequence encoding the polypeptide of interest.

The invention provides methods for introducing one or more heterologous nucleotide sequences into a duckweed plastome. The methods include the step of bombarding a duckweed tissue with a nucleic acid construct for chloroplast transformation where the nucleic acid construct is absorbed to a microprojectile and the bombardment takes place under conditions that promote the introduction of the nucleic acid construct into the plastid genome. In some embodiments, the heterologous nucleotide sequence is introduced into a duckweed chloroplast genome.

Methods for obtaining a transplastomic duckweed plant are provided. The methods include the steps of bombarding a duckweed tissue with a nucleic acid construct for chloroplast transformation that is absorbed to a microprojectile under conditions such that the nucleic acid construct for chloroplast transformation is introduced into at least one plastid of the duckweed tissue, and regenerating a duckweed plant from the duckweed tissue. In some embodiments, the nucleic acid construct includes a selectable marker sequence that confers a selectable phenotype to the duckweed tissue and the duckweed tissue is maintained in a selection medium after transformation, where the selection medium preferentially promotes the survival of duckweed cells having plastids that are transformed with the selectable marker sequence.

### BRIEF DESCRIPTION OF THE DRAWINGS

FIG. 1 shows a schematic diagram of the 16S to 23S rRNA region of the Lemna chloroplast genome. See the Experimental section for details.

FIG. 2 shows a schematic diagram of the expression vector pBCT01. See the Experimental section for details.

FIG. 3 shows the location of PCR primers 71, 248, 210, and 234 within the pBCT01 transgene region. These primers were used to confirm the integration of the pBCT01 transgene into duckweed chloroplasts. See the Experimental section for details.

FIG. 4 shows a schematic diagram of the expression vector pBCT04. See the Experimental section for details.

### DETAILED DESCRIPTION OF THE INVENTION

Plastids are organelles found in plant cells that carry out photosynthesis and play a major role in the biosynthesis of amino acids, complex carbohydrates, fatty acids, and pigments. There are several types of differentiated plant plastids including amyloplasts, chromoplasts, chloroplasts etioplasts, and leucoplasts. All of these plastids are derived from a undifferentiated precursor plastid, termed a proplastid. Chloroplasts are the most common plastids, and are the site of photosynthesis in the plant cell. Each photosynthetic plant cell contains multiple chloroplasts, typically for 50 to 100 per cell. Chloroplasts have their own genome, a circular DNA molecule termed a plastome. Each chloroplast contains multiple copies, typically 50-100 copies, of the plastome.

The plant plastome has become a target for the introduction of foreign transgenes because the expression of a transgene from transformed plastomes (termed transplastomes) has several advantages in comparison with transformation of plant nuclei. In particular, because there are multiple copies of the plastome in each chloroplast and multiple chloroplasts in each cell, high numbers of integrated transgenes can be obtained in each plant cell using plastome transformation methods. This can result in a higher level of transgene expression in comparison with the expression level of the same transgene integrated into a plant nuclear genome.

Because plant plastids are an attractive target for genetic engineering, the present invention provides methods and compositions for the transformation of duckweed plastids. The compositions and methods of the invention are useful in increasing the recombinant protein production capacity of the duckweed expression system. The compositions of the invention include transformed duckweed plastids and transplastomic duckweed cells and plants, as well as nucleic acid constructs useful for transforming duckweed plastids. The invention also provides methods for introducing one or more heterologous nucleotide sequences into a duckweed plastome. The compositions and methods of the invention are described in detail below.

Definitions:

The terms “expression” or “production” refer to the biosynthesis of a gene product, including the transcription, translation, and assembly of the gene product. The term “duckweed” refers to members of the family Lemnaceae. This family currently is divided into five genera and 38 species of duckweed as follows: genus Lemna (L. aequinoctialis, L. disperma, L. ecuadoriensis, L. gibba, L. japonica, L. minor, L. miniscula, L. obscura, L. perpusilla, L. tenera, L. trisulca, L. turionifera, L. valdiviana); genus Spirodela (S. intermedia, S. polyrrhiza); genus Wolffia (Wa. angusta, Wa. arrhiza, Wa. australina, Wa. borealis, Wa. brasiliensis, Wa. columbiana, Wa. elongata, Wa. globosa, Wa. microscopica, Wa. neglecta) genus Wolfiella (Wl. caudata, Wl. denticulata, Wl. gladiata, Wl. hyalina, Wl. lingulata, Wl. repunda, Wl. rotunda, and Wl. neotropica), and genus Landoltia (L. punctata). Any other genera or species of Lemnaceae, if they exist, are also aspects of the present invention. Lemna species can be classified using the taxonomic scheme described by Les et al. (2002) Systematic Botany 27:221-40.

The term “duckweed nodule” as used herein refers to duckweed tissue comprising duckweed cells where at least about 50%, 55%, 60%, 65%, 70%, 75%, 80%, 85%, 90%, 95%, or 100% of the cells are differentiated cells. A “differentiated cell,” as used herein, is a cell with at least one phenotypic characteristic (e.g., a distinctive cell morphology or the expression of a marker nucleic acid or protein) that distinguishes it from undifferentiated cells or from cells found in other tissue types. The differentiated cells of the duckweed nodule culture described herein form a tiled smooth surface of interconnected cells fused at their adjacent cell walls, with nodules that have begun to organize into frond primordium scattered throughout the tissue. The surface of the tissue of the nodule culture has epidermal cells connect to each other via plasmadesmata. “Operably linked” as used herein in reference to nucleotide sequences refers to multiple nucleotide sequences that are placed in a functional relationship with each other. Generally, operably linked DNA sequences are contiguous and, where necessary to join two protein coding regions, in reading frame. For example, an expression control sequence is operably linked to a nucleotide sequence encoding a polypeptide of interest when the expression control sequence is position such that it modulates the transcription of the nucleotide sequence encoding the polypeptide of interest. In another example, a targeting nucleotide sequence is operably linked to an expression cassette when it is positioned such that it can recombine homologously with a target site in a target genome sequence such that the expression cassette is transferred to the target genome sequence.

A nucleotide sequence that is “heterologous to a duckweed plastome” as used herein refers to a nucleotide sequence that is not native to the recipient plastome into which it integrates.

A. Plastids

The duckweed plastids to be transformed in the present invention may be from any cell type and may be in a differentiated or undifferentiated state. Examples of duckweed plastids that may be used include undifferentiated proplastids, amyloplasts, chromoplasts, chloroplasts, etioplasts, and leucoplasts.

Plastids comprise their own genome, termed a plastome. Typically, individual plastids comprise multiple plastomes, most typically 50 to 100 plastomes per plastid. Plastomes that may be transformed with a nucleic acid molecule of the invention are referred to as “recipient plastomes,” and recipient plastomes that have been transformed with a heterologous nucleotide sequence are referred to as “transplastomic.” In some embodiments, all of the plastomes of a transplastomic plastid are substantially identical, i.e. they all contain the coding region of the transforming nucleic acid molecule and preferably contain any associated expression control sequence, or at least enough of any expression control sequence to promote expression of the coding sequence.” Such transplastomic plastids, and cells and plants containing such plastids, are referred to as “homotransplastomic.”

In some embodiments, the transplastomic plastids are stably transformed with a heterologous nucleotide sequence. Stable transformation of a duckweed plastid is shown when the heterologous nucleotide sequence is detectable over a period of time, indicating that the heterologous nucleotide sequence is retained by the plastome. Methods of detecting recombination are well known in the art and include, for example, Southern analysis using probes that can detect the heterologous nucleotide sequence, or amplification of the transgene by PCR.

B. Plastid Transformation Vectors

1. Expression Cassettes

According to the present invention, transformed duckweed plastids may be obtained by transformation with a plastid transformation vector comprising a nucleotide sequence encoding a polypeptide of interest contained within an expression cassette. The expression cassette comprises an expression control sequence operably linked to the nucleotide sequence encoding the polypeptide of interest. In some embodiments of the invention, the nucleic acid molecule to be introduced into the duckweed plastid contains two or more expression cassettes, each of which encodes at least one polypeptide of interest.

The expression control sequence may comprise one or more promoter sequences, one or more enhancer sequences, or both promoter and enhancer sequences. The expression control sequence may be native to or foreign to the nucleotide sequence of interest. By “native,” it is intended that the expression control sequence is operably linked to the nucleotide sequence encoding the polypeptide of interest in a wild type genome. By “foreign,” it is intended that the expression control sequence is not operably linked to the nucleotide sequence encoding the polypeptide of interest in a wild type genome.

Expression control sequences may be derived from any organism so long as they are capable of promoting transcription of a sequence in a duckweed plastid. Examples of plant plastid promoters include, but are not limited to, the promoter from the psbA gene, the rbcL gene, or the atpB rRNA, as well as rRNA promoters such as the 16S rRNA promoter sequence from tobacco. See, Boyton et al. (1988) Nature 240:1534-1538; Daniell et al. (1990) Proc. Natl. Acad. Sci. USA 87:88-92; and Sriramn et al. (1998) Plant Physiol. 117:1495-1499; all of which are herein incorporated by reference in their entirety. Exemplary promoters also include, but are not limited to, those described in Zurawski et al. (1981) Nucleic Acids Res. 9:3251-3270; Zurawski et al. (1982) 79:7699-7703; Krebbers et al. (1982) Nucleic Acids Res. 10:4985-5002. Mullet et al. (1985) Plant Molec. Biol. 4:39-54; Hanley-Bowdoin and Chua (1989), Mol. Gen. Genet. 215:217-24; Svab et al. (1990) Proc. Natl. Acad. Sci. USA 87:8526-8530; and Svab and Maliga (1993) Proc. Natl. Acad. Sci. USA 90:913-17; herein incorporated in their entirety by reference. Expression control sequences of the invention may also be generated by recombinant techniques or by other synthetic means. See, for example, NCBI Accession Numbers. AJ276677 and ABA276676, herein incorporated by reference.

Expression control sequences may be chosen to give a desired level of regulation. For example, in some instances, it may be advantageous to use expression control sequences that are activated in response to specific environmental stimuli (e.g., heat shock gene promoters, drought-inducible gene promoters, pathogen-inducible gene promoters, wound-inducible gene promoters, and light/dark-inducible gene promoters) or plant growth regulators (e.g., promoters from genes induced by abscissic acid, auxins, cytokinins, and gibberellic acid). For example, expression control sequences of a gene whose expression is regulated by light may be used to confer light-regulated expression. Examples of such genes include SSU or chlorophyll A/B binding protein genes. See, for example, Shiina et al. (1997) Plant Physiol. 115:477-483, and Eilenberg et al. (1998) Planta 206:204-14; herein incorporated by reference in their entirety.

Other inducible promoters may also be used in the present invention. See, for example, U.S. Pat. No. 5,925,806, which is herein incorporated by reference in its entirety. This patent describes a system for inducing the expression of a transgene integrated within a plastid genome. The method comprises transforming the nuclei of the plant with a sequence encoding a viral RNA polymerase under the control of a constitutive or inducible promoter, and introducing a transgene into the plastid of this plant, where the transgene is operably linked to a promoter specific for the viral RNA polymerase. The expression of the plastid transgene can then be induced by inducing the expression of the viral RNA polymerase gene in the nucleus of the plant.

Other promoters that direct transcription in duckweed plastids may be identified by techniques known in the art. For example, the candidate promoter sequence may be inserted next to a marker sequence that lacks an expression control sequence and this expression cassette may then be used to transform a duckweed plastid. The level of expression of the marker sequence may then be used to determine the strength of the promoter in the duckweed plastid. Methods of increasing the efficiency of transcription of a nucleotide sequence are known in the art. Such methods include, for example, the use of multiple promoters inserted into the expression cassette in tandem and the addition of enhancer sequences to the expression cassette.

The expression cassette may also include transcription termination sequences. Any suitable termination sequence known in the art may be used in accordance with the present invention. The termination region may be native with the transcriptional initiation region, may be native with the nucleotide sequence of interest, or may be derived from another source. Exemplary transcription termination sequences include the psbA termination sequence and the rps16 termination sequence. Other suitable termination sequences will be apparent to those skilled in the art.

The expression cassette may contain more than one coding sequence targeted for integration into the plastome. In some embodiments, each nucleic acid sequence will be operably linked to one or more expression control sequences and transcription termination sequences. Alternatively, multiple expression cassettes may be provided.

The plastid transformation vector typically contains an expression cassette comprising a nucleotide sequence encoding a polypeptide of interest. The polypeptide of interest may be any polypeptide, for example insulin, growth hormone, α-interferon, β-interferon, β-glucocerebrosidase, β-glucoronidase, retinoblastoma protein, p53 protein, angiostatin, leptin, erythropoietin, granulocyte macrophage colony stimulating factor, plasminogen, monoclonal antibodies, fragment antigen binding (Fab) fragments, cytokines, receptors, human vaccines, animal vaccines, plant polypeptides, peptides, and serum albumin. The invention is not limited to the expression of any particular polypeptide of interest.

In some embodiments, the duckweed plastome is engineered such that multimeric proteins (e.g., monoclonal antibodies, hemoglobin, P450 oxidase, and collagen, and the like) are produced. One exemplary approach for producing biologically active multimeric proteins in duckweed uses a chloroplast transformation vector containing the genes encoding all of the polypeptide subunits. See, e.g., During et al. (1990) Plant Mol. Biol. 15:281 and van Engelen et al. (1994) Plant Mol. Biol. 26:1701. This vector is then introduced into duckweed plastomes using the methods described elsewhere herein. This method results in clonal duckweed cell or plant lines that express all of the polypeptides necessary to assemble the multimeric protein. In some instances, it may be desirable to produce less than all of the subunits of a multimeric protein, or even a single protein subunit, in a transformed duckweed plant or duckweed nodule culture, e.g., for industrial or chemical processes or for diagnostic, therapeutic, or vaccination purposes.

In some embodiments, plastid transformation vector includes an expression cassette contains a nucleotide sequence encoding a selectable marker polypeptide that can be used for the selection of transformed cells or tissues. Marker polypeptides include those conferring antibiotic resistance, as well as those conferring resistance to herbicidal compounds. Herbicide resistance genes generally code for a modified target protein insensitive to the herbicide or for an enzyme that degrades or detoxifies the herbicide in the plant before it can act. See DeBlock et al. (1987) EMBO J. 6:2513; DeBlock et al.(1989) Plant Physiol. 91:691; Fromm et al. (1990) BioTechnology 8:833; Gordon-Kamm et al. (1990) Plant Cell 2:603. For example, resistance to glyphosphate or sulfonylurea herbicides has been obtained using genes coding for the mutant target enzymes, 5-enolpyruvylshikimate-3-phosphate synthase (EPSPS) and acetolactate synthase (ALS). Resistance to glufosinate ammonium, boromoxynil, and 2,4-dichlorophenoxyacetate (2,4-D) have been obtained by using bacterial genes encoding phosphinothricin acetyltransferase, a nitrilase, or a 2,4-dichlorophenoxyacetate monooxygenase, which detoxify the respective herbicides.

For purposes of the present invention, marker sequences include, but are not limited to, nucleotide sequences encoding aminoglycoside 3′-adenylyltransferase (see, for example, Svab and Maliga (1993) Proc. Natl. Acad. Sci. USA 90:913-917); neomycin phosphotransferase II (Fraley et al. (1986) CRC Critical Reviews in Plant Science 4:1); neomycin phosphotransferase III; cyanamide hydratase (Maier-Greiner et al. (1991) Proc. Natl. Acad. Sci. USA 88:4250); aspartate kinase; dihydrodipicolinate synthase (Perl et al. (1993) BioTechnology 11:715); bar gene (Toki et al. (1992) Plant Physiol. 100:1503; Meagher et al. (1996) Crop Sci. 36:1367); tryptophan decarboxylase (Goddijn et al. (1993) Plant Mol. Biol. 22:907); neomycin phosphotransferase (NEO; Southern et al. (1982) J Mol. Appl. Gen. 1:327); hygromycin phosphotransferase (HPT or HYG; Shimizu et al. (1986) Mol. Cell. Biol. 6:1074); dihydrofolate reductase (DHFR; Kwok et al. (1986) Proc. Natl. Acad. Sci. USA 83:4552); phosphinothricin acetyltransferase (DeBlock et al. (1987) EMBO J 6:2513); 2,2-dichloropropionic acid dehalogenase (Buchanan-Wollatron et al. (1989) J Cell. Biochem. 13D:330); acetohydroxyacid synthase (U.S. Pat. No. 4,761,373 to Anderson et al.; Haughn et al. (1988) Mol. Gen. Genet. 221:266); 5-enolpyruvyl-shikimate-phosphate synthase (aroA; Comai et al. (1985) Nature 317:741); haloarylnitrilase (WO 87/04181 to Stalker et al.); acetyl-coenzyme A carboxylase (Parker et al. (1990) Plant Physiol. 92:1220); dihydropteroate synthase (sulI; Guerineau et al. (1990) Plant Mol. Biol. 15:127); and 32 kDa photosystem II polypeptide (psbA; Hirschberg et al. (1983) Science 222:1346 (1983).

Also included are genes encoding resistance to: gentamycin (e.g., aacC1, Wohlleben et al. (1989) Mol. Gen. Genet. 217:202-208); chloramphenicol (Herrera-Estrella et al. (1983) EMBO J. 2:987); methotrexate (Herrera-Estrella et al. (1983) Nature 303:209; Meijer et al. (1991) Plant Mol. Biol. 16:807); hygromycin (Waldron et al. (1985) Plant Mol. Biol. 5:103; Zhijian et al. (1995) Plant Science 108:219; Meijer et al. (1991) Plant Mol. Bio. 16:807); streptomycin (Maliga et al. (1973) Nature 255:401-402; Etzold et al. (1987) FEBS Lett. 219:343-346; and Fromm et al. (1989) Plant Mol. Biol. 12:499-505) spectinomycin (Fromm et al. (1987) EMBO J 6:3233-3237); lincomycin (Cseplo and Maliga (1984) Mol. Gen. Genet. 196:407-412; Cseplo et al. (1988) Mol. Gen. Genet. 214:295-299; bleomycin (Hille et al. (1986) Plant Mol. Biol. 7:171); sulfonamide (Guerineau et al. (1990) Plant Mol. Bio. 15:127); bromoxynil (Stalker et al. (1988) Science 242:419); 2,4-D (Streber et al. (1989) BioTechnology 7:811); phosphinothricin (DeBlock et al. (1987) EMBO J. 6:2513); spectinomycin (Bretagne-Sagnard and Chupeau, Transgenic Research 5:131).

The bar gene confers herbicide resistance to glufosinate-type herbicides, such as phosphinothricin (PPT) or bialaphos, and the like. As noted above, other selectable markers that could be used in the vector constructs include, but are not limited to, pat, for bialaphos and phosphinothricin resistance, ALS for imidazolinone resistance, HPH or HYG for hygromycin resistance, EPSP synthase for glyphosate resistance, Hm 1 for resistance to the Hc-toxin, protophoryinogen oxidase and variants thereof for resistant to diphenyl ether (DPE) herbicides such as oxyfluorfen, and other selective agents used routinely and known to one of ordinary skill in the art. See Yarranton (1992) Curr. Opin. Biotech. 3:506; Chistopherson et al. (1992) Proc. Natl. Acad. Sci. USA 89:6314; Yao et al. (1992) Cell 71:63; Reznikoff (1992) Mol. Microbiol. 6:2419; Barkley et al. (1980) The Operon 177-220; Hu et al. (1987) Cell 48:555; Brown et al. (1987) Cell 49:603; Figge et al. (1988) Cell 52:713; Deuschle et al. (1989) Proc. Natl. Acad Sci. USA 86:5400; Fuerst et al. (1989) Proc. Natl. Acad. Sci. USA 86:2549; Deuschle et al. (1990) Science 248:480; Labow et al. (1990) Mol. Cell. Biol. 10:3343; Zambretti et al. (1992) Proc. Natl. Acad. Sci. USA 89:3952; Baim et al. (1991) Proc. Natl. Acad. Sci. USA 88:5072; Wyborski et al. (1991) Nuc. Acids Res. 19:4647; Hillenand-Wissman (1989) Topics in Mol. And Struc. Biol. 10:143; Degenkolb et al. (1991) Antimicrob. Agents Chemother. 35:1591; Kleinschnidt et al. (1988) Biochemistry 27:1094; Gatz et al. (1992) Plant J. 2:397; Gossen et al. (1992) Proc. Natl. Acad. Sci. USA 89:5547; Oliva et al. (1992) Antimicrob. Agents Chemother. 36:913; Hlavka et al. (1985) Handbook of Experimental Pharmacology 78; Gill et al. (1988) Nature 334:721, U.S. Pat. Nos. 6,282,837, 6,288,306, and 5,767,373, PCT publication WO 0112825, and U.S. Pat. publication 20010016956. Such disclosures are herein incorporated by reference.

Another example of a marker polypeptide for use in the present invention is betaine aldehyde dehydrogenase (BADH). This enzyme catalyzes the conversion of toxic betaine aldehyde to non-toxic glycine betaine, an osmoprotectant. See, Daniell H. et al. (2001) Current Genetics 39:109-16).

The above list of selectable marker sequence is not meant to be limiting. Any selectable marker sequence can be used in the present invention.

The present invention provides for the modification of the expressed nucleotide sequences to enhance its expression in duckweed. One such modification is the synthesis of the nucleotide sequence of interest using plastid-preferred codons. For example, it has been shown that in some cases a transgene having an adenine/thymine content of greater than 50% is expressed more efficiently in a plastid than a transgene having a lower adenine/thymine content. See, for example, U.S. Pat. No. 5,545,817, herein incorporated by reference. Methods are available in the art for synthesizing nucleotide sequences with altered codon usage. See, for example, U.S. Pat. Nos. 5,380,831 and 5,436,391; Perlak et al. (1991) Proc. Natl. Acad. Sci. USA 15:3324; Iannacome et al. (1997) Plant Mol. Biol. 34:485; and Murray et al., (1989) Nucleic Acids. Res. 17:477, herein incorporated by reference. It is further recognized that all or any part of the gene sequence may be optimized or synthetic. In other words, fully optimized or partially optimized sequences may also be used. Other modifications can also be made to the nucleotide sequence of interest to enhance its expression in duckweed. These modifications include, but are not limited to, elimination of sequences encoding spurious polyadenylation signals, transposon-like repeats, and other such well characterized sequences which may be deleterious to gene expression. When possible, the sequence may be modified to avoid predicted hairpin secondary mRNA structures.

2. Targeting Nucleotide Sequences

The plastid transformation vectors of the invention comprise a first and second targeting nucleotide sequence, each of which is homologous to a duckweed plastome sequence. These targeting nucleotide sequences flank the expression cassette and define the 5′ and 3′ ends of the region of the plastid transformation vector to be transferred to the plastome. The targeting nucleotide sequences allow for the insertion of the expression cassette into the recipient plastome by homologous recombination.

The targeting sequences are homologous to regions of the recipient plastomes. Typically, the targeting nucleotide sequences that flank the expression cassette are at least 80%, at least 85%, at least 90%, at least 95%, at least 99%, or 100% identical to the targeted regions of the recipient plastome and are capable of recombining homologously with the recipient plastome.

The comparison of sequences and determination of percent identity and percent similarity between two sequences can be accomplished using a mathematical algorithm. In the present invention, the percent identity between two nucleotide sequences is determined using the GAP program in the GCG software package, using a BLOSUM62 scoring matrix (see Henikoff et al. (1989) Proc. Natl. Acad. Sci. USA 89:10915) and a gap weight of 40, 50, 60, 70, or 80 and a length weight of 1, 2, 3, 4, 5, or 6.

The first and second targeting nucleotide sequences may be homologous to the same, overlapping, coterminous, or distinct regions of the recipient plastome. The targeting sequences may be homologous to any region of the recipient plastome, for example regions comprising a gene, a pseudogene, or an intergenic sequence. The targeting sequences may be designed so that the nucleotide sequence encoding a polypeptide of interest is integrated in the plastome such that it is operably linked to one or more expression control sequences native to the duckweed plastome. In this embodiment, the native expression control sequence can be used to drive the expression of the nucleotide sequence encoding the polypeptide of interest. In another embodiment, either or both of the targeting nucleotide sequence may contain one or more expression control sequences that can be used to drive the expression of the nucleotide sequence encoding the polypeptide of interest.

The targeting nucleotide sequences may be any length that is sufficient to allow for homologous recombination with the recipient plastome. Targeting nucleotide sequences containing nucleotide sequences less than 100 nucleotides in length may be sufficient to allow recombination in some cases. In other embodiments, longer targeting nucleotide sequences may provide for more efficient homologous recombination. Such targeting nucleotide sequences may be at least 200, 300, 400, 500, 600, 700, 800, or 900 nucleotides, or at least 1000 or more nucleotides in length.

The present invention provides the sequence of the 16S to 23 S rRNA regions of the Lemna minor chloroplast genome. This nucleotide sequence is shown in SEQ ID NO:3. In some embodiments, the targeting nucleotide sequences are homologous to at least 200, 300, 400, 500, 600, 700, 800, or 900 nucleotides, or at least 1000, 1500, 2000, 2500, or 3000 or more nucleotides of the sequences. However, the targeting nucleotide sequences of the invention may be homologous to any duckweed plastome sequence. Additional non-limiting examples of intergenic regions that may be used as targeting sequences include the tRNAGlu (trnE) to tRNAThr (trnT) region, the trnE to tRNATyr (trnY), the trnY to tRNAAsp (trnD) region, the rps8 to rps14 region, the ribulose-1,5-bisphosphate carboxylase/oxygenase large subunit (rbcL) to β subunit of acetyl-coenzyme A carboxylase (accD) region, the large subunit polypeptide of cytochrome b(559) (psbE) to cytochrome f (petA) region, the tRNAGly (trnG) to tRNAMet (trnM) region, the psbA to tRNAHis (trnH) region, the tRNA Val (trnV) to 16srRNA region, the trnV to rps7/rps12 region, and regions within non-essential genes of a duckweed plastome.

C. Transformation Methods

The transplastomic duckweed cells and plants of the invention are produced by transforming a duckweed plastid with a nucleotide sequence heterologous to the duckweed plastome. The heterologous nucleotide sequence may be inserted by random insertion, or site-directed integration (e.g., homologous recombination). The heterologous nucleotide sequence may be inserted into an isolated plastid or transformed into a plastid in vivo. The transformed plastid may be used to express the heterologous nucleotide sequence either in vivo or ex vivo.

Transformation of the plastid may be achieved by any suitable transformation method, for example, by electroporation of plant protoplasts (see, Taylor and Walbot (1985) Proc. Natl. Acad. Sci. USA 82:5824-28), PEG-based procedures (see, Golds et al. Biotechnology 11:95-97, microinjection (see, Neuhas et al. (1987) Theor. Appl. Genet. 74:30-36), or by particle bombardment (see, Boynton et al. (1988) Science 240:1534-38; Svab et al. (1990) Proc. Natl. Acad. Sci. 87:8526-8530; Svab and Maliga (1993) Proc. Natl. Acad. Sci. 90:913-917; and U.S. Pat. Nos. 5,451,513, 5,545,817, 5,545,818, 5,576,198, and 5,866,421; all of which are herein incorporated in their entirety by reference).

Transformation of duckweed nuclei by particle bombardment has been described in U.S. Pat. No. 6,040,498, herein incorporated by reference. In embodiments of the present invention, the ballistic transformation method comprises the steps of: (a) providing a duckweed tissue as a target; (b) propelling the microprojectile carrying the chloroplast transformation vector at the duckweed tissue at a velocity sufficient to pierce the walls of the cells within the tissue and to deposit the nucleotide sequence within a cell of the tissue to thereby provide a transformed tissue. In particular embodiments of the invention, the method further includes the step of culturing the transformed tissue with a selection agent, as described below. In a further alternate embodiment, the selection step is followed by the step of regenerating transformed duckweed plants from the transformed tissue.

Any ballistic cell transformation apparatus can be used in practicing the present invention. Exemplary apparatuses are disclosed by Sanford et al. (1988) Particulate Science and Technology 5:27; Klein et al. (1987) Nature 327:70, and in U.S. Pat. Nos. 4,945,050, 5,036,006, 5,100,792, 5,179,022, 5,204,253, 5,371,015, and 5,478,744; all of which are hereby incorporated by reference in their entirety. Such apparatus typically allow for the control of the pressure used in bombardment. The pressure used in the present methods is typically between about 500 psi and about 2500 psi, for example from about 1100 psi to about 2200 psi.

The nucleotide sequence may be immobilized on the microcarrier particle by precipitation. The precise precipitation parameters employed will vary depending upon factors such as the particle acceleration procedure employed, as is known in the art. The carrier particles may optionally be coated with an encapsulating agents such as polylysine to improve the stability of nucleotide sequences immobilized thereon, as discussed in EP 0 270 356. Any microcarrier may be used in the present invention, including gold and tungsten. The microcarrier size is generally between about 0.6 μm and about 1.6 μm.

The tissue to be bombarded may be any duckweed cell or tissue, including duckweed fronds, duckweed nodules, fragmented duckweed nodules (micronodules), and partially regenerated nodules. Methods for producing duckweed nodules, micronodules, and partially regenerated nodules are described in U.S. Pat. No. 6,040,498 and U.S. patent application Ser. Nos. 09/915,873 and 10/158,243, all of which are hereby incorporated by reference in their entirety.

Transplastomic duckweed cells or nodule tissue may be regenerated into transplastomic or homotransplastomic plants. Methods of regenerating duckweed plants from transformed duckweed tissue have been described in U.S. Pat. No. 6,040,498 and in U.S. patent application Ser. Nos. 09/915,873 and 10/158,243, all of which are herein incorporated in their entirety by reference. Methods for regenerating transformed duckweed fronds from nodule culture are also described elsewhere herein.

A variety of selection protocols may be used in the present invention. For example, in some embodiments the transformed fronds or nodules may maintained in darkness for part of the selection process and then moved into light. In other embodiments, the fronds or nodules are maintained in light throughout the selection process.

In some embodiments, the transformed fronds or nodules, selection occurs on media which will maintain an undifferentiated state, and the plants are then moved to a media that induces frond regeneration, while in other embodiments the entire selection process occurs on media that induces frond regeneration.

In some embodiments, the transformed duckweed plastids and transplastomic duckweed plants and cells are homoplastomic. Homoplastomic cells and plants may be produced by transforming the duckweed plastid with a nucleotide sequence encoding a selectable marker polypeptide and then maintaining the transplastomic duckweed plant or cell in a selection medium that allows the survival of duckweed cells that express the marker polypeptide. In one embodiment, the selection medium allows for the growth of only those duckweed cells that are homoplastomic or have substantially all plastids transformed with the nucleotide sequence encoding the selectable marker polypeptide or are homoplastomic for the nucleotide sequence encoding the selectable marker polypeptide. Multiple rounds of selection may be required to produce duckweed plants or cells that are homoplastomic or have substantially all plastids transformed with the sequence encoding the marker polypeptide. Southern analysis or PCR analysis may be performed to determine if a transplastomic duckweed cell or plant is homoplastomic.

In some embodiments, selection for the marker phenotype conferred by the marker polypeptide allows for the selection of duckweed cells having a second coding sequence to which the sequence encoding the marker polypeptide has been linked. This second coding sequence typically encodes a polypeptide of interest, and the selected duckweed cells may be used to produce this polypeptide of interest.

### EXPERIMENTAL

The following examples are offered for purposes of illustration, not by way of limitation.

Lemna Chloroplast Transformation Vectors

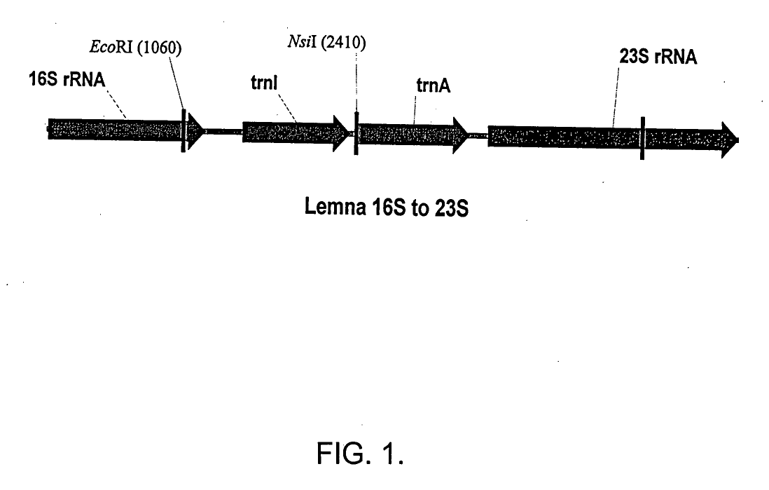

Vectors for the transformation of Lemna chloroplasts were constructed. The vectors included sequences designed for homologous recombination with sites in the 16S to 23S rRNA region of the Lemna chloroplast genome (see FIG. 1). The sequences used to promote homologous recombination with the Lemna chloroplast genome were identified as follows. The 16S to 23S rRNA regions from the chloroplast genomes of tobacco, maize, rice, Arabidopsis, and spinach were aligned to determine regions having a high level of sequence identity. Based on the conserved regions identified in the alignment, PCR primers were designed to amplify the 16S to 23S region of the Lemna chloroplast genome. The sequences of these PCR primers are shown below.

| | |
|---|---|
| Primer 167: | |
| 5′ GCTGGTCCGAGAGGATGATC 3′ | (SEQ ID NO:1) |
| Primer 166: | |
| 5′ GTTATAGTTACGGCCGCCGT 3′ | (SEQ ID NO:2) |

PCR was performed on Lemna minor genomic DNA using standard conditions. The resulting 5.4 kb PCR product was cloned into the pT7blue vector (Novagen®, Madison, Wis.), to form the plasmid pBCT00. The entire PCR product was then sequenced and was confirmed to be the 16S to 23S region of the Lemna chloroplast genome. The sequence of the PCR product is shown in SEQ ID NO:3. This region of the Lemna genome contains the 16S rRNA gene, tRNA isoleucine gene (trnI), tRNA alanine gene (trnA), and 23S rRNA gene.

The vectors were designed to target the integration of the transgene to the intergenic region between the trnI and trnA genes. Lemna chloroplast transformation vectors comprising a transgene flanked by homologous regions for recombination into the Lemna chloroplast genome and a selectable marker cassette was constructed as follows. Based on the Lemna genomic sequence obtained as described above, PCR primers were designed to amplify a 2.5 kb fragment from the Lemna 16S to 23S rRNA region. The sequences of these primers are shown below.

| | |
|---|---|
| Primer 207: | |
| 5′ ACAAGGTAGCCGTACTGGAAGGTGCGGCTG 3′ | (SEQ ID NO:4) |
| Primer 203: | |
| 5′ TTCAACTCCCCGAAGCATTTCGTC 3′ | (SEQ ID NO:5) |

PCR was performed on Lemna minor genomic DNA using standard conditions. The PCR product was amplified and cloned into pT7blue vector (Novagen®, Madison, Wis.).

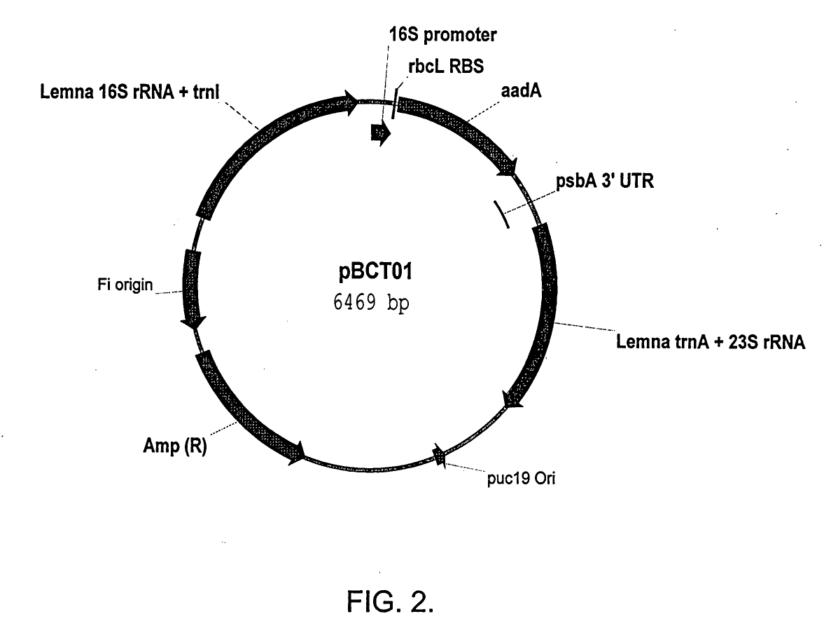

To produce vector pBCT01, a spectinomycin/streptomycin selectable marker cassette (NCBI Accession Number AF061065) encompassing the tobacco 16S rRNA promoter (shown in SEQ ID NO:6) with the RbcL ribosome binding site (shown in SEQ ID NO:7), aminoglycoside 3″-adenylyltransferase (aadA) gene, and tobacco psbA 3′UTR (shown in SEQ ID NO:8) was cloned with PstI/NsiI was inserted into pBCT00 in the intergenic region between the Lemna trnI and trnA, to produce the plasmid. A schematic diagram of plasmid pCBT04 is shown in FIG. 2.

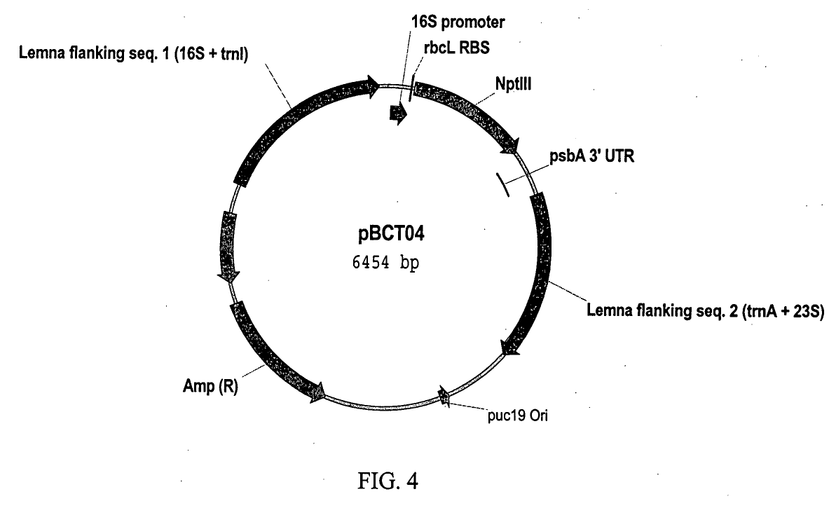

To produce vector pBCT04, a kanamycin selectable marker cassette encompassing the tobacco 16S rRNA promoter (shown in SEQ ID NO:6) with the RbcL ribosome binding site (shown in SEQ ID NO:7), aminoglycoside 3″-adenylyltransferase (aadA) gene, and tobacco psbA 3′UTR (shown in SEQ ID NO:8) was cloned with PstI/NsiI was inserted into pBCT00 in the intergenic region between the Lemna trnI and trnA, to produce the plasmid. The kanamycin selectable marker cassette was created by PCR amplifying the gene encoding aminoglycoside 3′ phosphotransferase (NCBI Accession No. P00554) and ligating it downstream of the tobacco 16SrRNA promoter with RbcL binding site and upstream of the tobacco psbA 3′ UTR. This cassette was cloned into the PstI site of the Leman TrnI and trnA intergenic region to create the vector pBCT04. A schematic diagram of plasmid pBCT04 is shown in FIG. 4.

Preparation of Lemna Nodules

In these examples, Lemna minor strain 8627 was used for transformation although any Lemna strain can be used. Duckweed nodule cultures for transformation were prepared as follows. Duckweed fronds were separated, the roots were cut off with a sterile scalpel, and the fronds are placed, ventral side down, on Murashige and Skoog medium (MS medium; Murashige and Skoog (1962) Physiol. Plant. 15:473) pH 5.6, supplemented with 5 μM 2,4-dichlorophenoxyacetic acid, 0.5 μM 1-Phenyl-3(1,2,3-thiadiazol-5-yl) urea thidiazuron (Sigma P6186), 3% sucrose, 0.4 Difco Bacto-agar (Fisher Scientific), and 0.15% Gelrite (Sigma). Fronds were grown for 5-6 weeks. At this time, the nodules (small, yellowish cell masses) appeared, generally from the central part of the ventral side. This nodule tissue pieces (average size 3-6 mm in diameter) were detached from the mother frond and cultured in MS medium supplemented with 3% sucrose, 0.4% Difco Bacto-agar, 1 μM 2,4-dichlorophenoxyacetic acid, and 2 μM benzyladenine.

Chloroplast Transformation of Lemna Nodules by Particle Bombardment:

Purified pBCT01 and pBCT04 DNA was prepared using a QIAGEN Maxi-Prep kit (QIAGEN, Valencia, Calif.) and gold microcarriers (0.6 μm; Bio-Rad Laboratories, Hercules, Calif.) were coated with the purified DNA according to the manufacturer's instructions. Prior to particle bombardment, approximately 50 Lemna nodules (approximately 0.5-10 mm in diameter) were placed in the center of a petri plate containing MS medium (for pBCT01) or MS medium supplemented with 5 μM 2,4-dichlorophenoxyacetic acid, 0.5 μM 1-Phenyl-3(1,2,3-thiadiazol-5-yl) urea thidiazuron, 3% sucrose, 0.4 Difco Bacto-agar, and 0.15% Gelrite (for pBCT04). The nodules were bombarded two times using a Bio-Rad PDS-1000/He biolistic instrument from Bio-Rad (PDS-1000/He, Bio-Rad, Hercules, Calif.) following the manufacturers protocol at a target distance of 9 cm, helium pressures ranging from 1100 to 2200, and a vacuum of 28 inches.

For pBCT01, the nodules were incubated in the dark on MS medium for 48 hours after bombardment. The nodules were then transferred to MS medium containing 25 μg/ml spectinomycin dihydrochloride and grown under continuous light for 2 weeks (for pBCT01). The nodules were then transferred to 0.5×SH medium containing 25 μg/ml spectinomycin and grown under continuous light. The nodules were transferred to fresh medium once a week. Nodules began to generate frond tissue after 6 to 8 weeks on 0.5×SH medium.

For pBCT04, the nodules were incubated in the dark on MS medium supplemented with 5 μM 2,4-dichlorophenoxyacetic acid, 0.5 μM 1-Phenyl-3(1,2,3-thiadiazol-5-yl) urea thidiazuron, 3% sucrose, 0.4 Difco Bacto-agar, and 0.15% Gelrite for 48 hours after bombardment. The nodules were then transferred to MS medium supplemented with 5 μM 2,4-dichlorophenoxyacetic acid, 0.5 μM 1-Phenyl-3(1,2,3-thiadiazol-5-yl) urea thidiazuron, 3% sucrose, 0.4 Difco Bacto-agar, 0.15% Gelrite, and 50 μg/ml kanamycin and maintained for four weeks, and then to MS medium supplemented with 5 μM 2,4-dichlorophenoxyacetic acid, 0.5 μM 1-Phenyl-3(1,2,3-thiadiazol-5-yl) urea thidiazuron, 3% sucrose, 0.4 Difco Bacto-agar, 0.15% Gelrite, and 100 μg/ml kanamycin for an additional 4 weeks. Nodules were then placed on 0.5×SH medium with 100 μg/ml kanamycin for 8-12 weeks until fronds were regenerated.

Chloroplast Transformation of Lemna Fronds by Particle Bombardment:

For transformation of Lemna fronds, approximately 30 Lemna fronds were placed in the center of a piece of # 5 Whatman paper on 0.5×SH media and bombarded as described above for Lemna nodules. After bombardment, the fronds were incubated in the dark for on 0.5×SH media for 48 hours, and then transferred to Murashige and Skoog medium supplemented with 3% sucrose, 0.4% Difco Bacto-agar, 0.15% Gelrite, 1 μM 2,4-dichlorophenoxyacetic acid, 2 μM benzyladenine, and 50 μg/ml kanamycin. Fronds were transferred to fresh media weekly for 6 to 8 weeks until nodule tissue was produced. Nodule tissue was transferred to 0.5×SH with 100 μg/ml kanamycin and transferred to fresh media weekly for 6 to 10 weeks until frond regeneration.

Analysis of Chloroplast Transformants

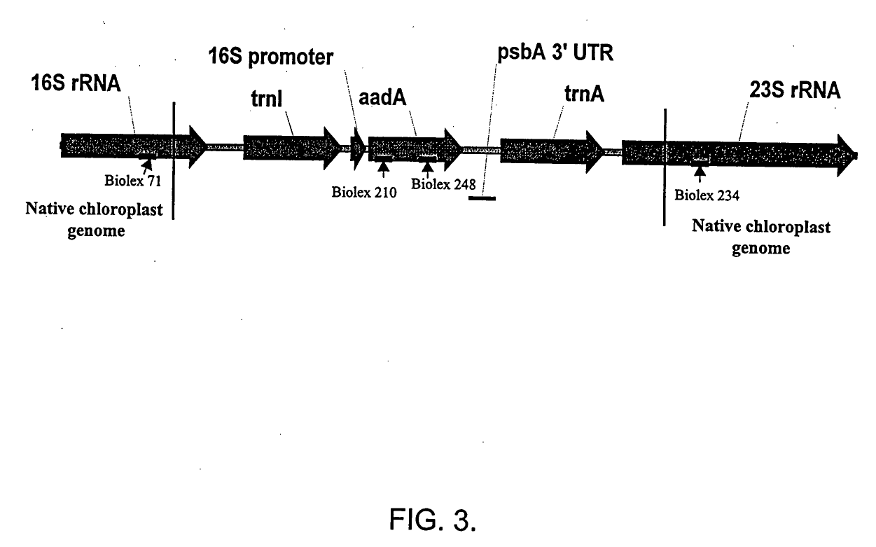

In order to test for the integration of the pBCT01 transgene region into the Lemna chloroplast genome, PCR was performed on nodule tissues that were generating fronds. Tissue was taken for analysis after 8 weeks of growth on SH medium with 25 μg/ml spectinomycin. Genomic DNA was extracted from the nodule tissue using a DNeasy DNA extraction kit (QIAGEN, Valencia, Calif.) and PCR was performed using standard conditions. Two primer sets were designed to test for integration on both sides of the homologous recombination site. Primers 71 and 248 tested for integration on the 16S side of the recombination site and amplified a 1796 bp product while primers 210 and 234 tests on the 23S side making a 2610 bp product. The primer locations are shown in FIG. 3.

| | |
|---|---|
| Primer 71: | |
| 5′ AAAACCCGTCCTCAGTTCGGATTGC 3′ | (SEQ ID NO:9) |
| Primer 248: | |
| 5′ CCGCGTTGTTTCATCAAGCCTTACG 3′ | (SEQ ID NO:10) |
| Primer 210: | |
| 5′ CTGTAGAAGTCACCATTGTTGTGC 3′ | (SEQ ID NO:11) |
| Primer 234: | |
| 5′ GGTTCGGACCTCCACTTAGT 3′ | (SEQ ID NO:12) |

The expected PCR products were produced in all six of the independent duckweed cultures, with two of these samples showing particularly high levels of transgene integration. The PCR product isolated from one of these cultures was sequenced and was shown to be identical to the pBCT01 transgene sequence, demonstrating homologous recombination between the transgene and the plastome.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. A duckweed plant having plastids that are transformed with at least one nucleotide sequence heterologous to a duckweed plastome, wherein the nucleotide sequence heterologous to a duckweed plastome is operably linked to an expression control sequence that is capable of functioning in the plastid.

2. The duckweed plant of claim 1, wherein at least one nucleotide sequence heterologous to the duckweed plastome encodes a polypeptide of interest.

3. The duckweed plant of claim 2, wherein said polypeptide of interest is selected from the group consisting of insulin, growth hormone, α-interferon, β-interferon, β-glucocerebrosidase, β-glucoronidase, retinoblastoma protein, p53 protein, angiostatin, leptin, erythropoietin, granulocyte macrophage colony stimulating factor, plasminogen, monoclonal antibodies, Fab fragments, single chain antibodies, cytokines, receptors, human vaccines, animal vaccines, plant polypeptides, peptides, and serum albumin.

4. The duckweed plant of claim 1, wherein said plastids are transformed with at least two nucleotide sequences heterologous to the duckweed plastome that encode polypeptides of interest.

5. The duckweed plant of claim 1, wherein said nucleotide sequence heterologous to the duckweed plastome comprises a coding sequence for a marker polypeptide that can be used for the selection of plant cells.

6. The duckweed plant of claim 5, wherein said marker polypeptide confers resistance to at least one antibiotic selected from the group consisting of spectinomycin and streptomycin.

7. The duckweed plant of claim 1, wherein the plastids that are transformed with at least one nucleotide sequence heterologous to a duckweed plastome are selected from the group consisting of proplastids, amyloplasts, chromoplasts, chloroplasts, etioplasts or leucoplasts.

8. The duckweed plant of claim 9, wherein the plastids that are transformed with at least one nucleotide sequence heterologous to a duckweed plastome are chloroplasts.

9. The duckweed plant of claim 1, wherein the expression control sequence functional in the plastid is a plant 16S rRNA promoter sequence.

10. The duckweed plant of claim 9, wherein the expression control sequence functional in the plastid is a tobacco 16S rRNA promoter sequence.

11. The duckweed plant of claim 10, wherein said tobacco 16S rRNA promoter sequence is the nucleotide sequence set forth in SEQ ID NO:6.

12. The duckweed plant of claim 1, wherein said duckweed plant is selected from the group consisting of the genus Spirodela, genus Wolffia, genus Wolfiella, and genus Lemna.

13. The duckweed plant of claim 12, wherein said duckweed plant is selected from the group consisting of Lemna minor, Lemna miniscula, Lemna aequinoctialis, and Lemna gibba.

14. A nucleic acid molecule comprising, as operably linked components, the following nucleotide sequences:
a) a first targeting nucleotide sequence homologous to a duckweed plastome sequence;
b) the nucleotide sequence of an expression cassette comprising at least one nucleotide sequence encoding a polypeptide of interest wherein said nucleotide sequence encoding a polypeptide of interest is heterologous to a duckweed plastome; and
c) a second targeting nucleotide sequence homologous to a duckweed plastome sequence.

15. The nucleic acid molecule of claim 14, wherein said expression cassette additionally comprises an expression control sequence operably linked to said nucleotide sequence encoding a polypeptide of interest.

16. The nucleic acid molecule of claim 14 wherein the expression control sequence functional in the plastid is a tobacco 16S rRNA promoter sequence.

17. The duckweed plant of claim 16 wherein said tobacco 16S rRNA promoter sequence is the nucleotide sequence set forth in SEQ ID NO:6.

18. The nucleic acid molecule of claim 14, additionally comprising at least one ribosome binding site nucleotide sequence that is functional in duckweed.

19. The nucleic acid molecule of claim 18 wherein said ribosome binding site that is functional in duckweed comprises the tobacco rbcL ribosome binding site nucleotide sequence set forth in SEQ ID NO:7.

20. The nucleic acid molecule of claim 14, additionally comprising a transcription termination sequence.

21. The nucleic acid molecule of claim 20 wherein said transcription termination sequence is the tobacco psbA gene 3′ untranslated region nucleotide sequence set forth in SEQ ID NO:8.

22. The nucleic acid molecule of claim 14, wherein at least one polypeptide of interest is a marker polypeptide capable of being used to select for transformed duckweed cells.

23. The nucleic acid molecule of claim 22 wherein the marker polypeptide confers resistance to an antibiotic selected from streptomycin and spectinomycin.

24. The nucleic acid molecule of claim 14 wherein said first and second targeting nucleotide sequences are capable of promoting homologous recombination within a duckweed plastome.

25. The nucleic acid molecule of claim 14 wherein said duckweed plastid is a duckweed chloroplast and said duckweed plastome is a chloroplast genome.

26. A duckweed chloroplast comprising the nucleic acid molecule of claim 14.

27. A duckweed cell comprising the chloroplast of claim 26.

28. A duckweed plant comprising the duckweed cell of claim 27.

29. The nucleic acid molecule of claim 14, wherein at least one nucleotide sequence selected from the first targeting nucleotide sequence and the second targeting nucleotide sequence is homologous to the nucleotide sequence set forth in SEQ ID NO:3.

30. A nucleic acid molecule comprising, as operably linked components, the following nucleotide sequences:
a) a first targeting nucleotide sequence homologous to a duckweed chloroplast genome sequence;
b) an expression control sequence functional in duckweed;
c) a ribosome binding site nucleotide sequence that is functional in duckweed;
d) a nucleotide sequence heterologous to a duckweed chloroplast genome, wherein said nucleotide sequence heterologous to a duckweed chloroplast genome encodes a polypeptide of interest;
e) a transcription termination sequence that is functional in duckweed; and
f) a second targeting nucleotide sequence homologous to a duckweed chloroplast genome sequence.

31. The nucleic acid molecule of claim 30, wherein at least one nucleotide sequence selected from the first targeting nucleotide sequence and the second targeting nucleotide sequence is homologous to the nucleotide sequence set forth in SEQ ID NO:3.

32. A duckweed chloroplast comprising the nucleic acid molecule of claim 30.

33. A duckweed cell comprising the chloroplast of claim 32.

34. A duckweed plant comprising the duckweed cell of claim 33.

35. A method for introducing at least one nucleotide sequence heterologous to a duckweed plastome into a duckweed plastid, said method comprising bombarding a duckweed nodule with a nucleic acid molecule of claim 14 absorbed to a microprojectile under conditions such that said nucleotide sequence heterologous to a duckweed plastome is introduced into at least one plastid of said duckweed nodule.

36. The method of claim 35, wherein said duckweed plastid is a duckweed chloroplast.

37. A duckweed plastid comprising at least one nucleotide sequence heterologous to a duckweed plastome wherein said duckweed plastid comprising at least one nucleotide sequence heterologous to a duckweed plastome is produced by the method of claim 35.

38. A duckweed cell containing the duckweed plastid of claim 37.

39. A duckweed plant containing the duckweed cell of claim 38.

40. A method for obtaining a transplastomic duckweed plant, said method comprising the steps of:
a) bombarding a duckweed nodule with a nucleic acid molecule of claim 14 absorbed to a microprojectile under conditions such that said nucleotide sequence heterologous to a duckweed plastome is introduced into at least one plastid of said duckweed nodule; and
b) regenerating a duckweed plant from said duckweed nodule.

41. A transplastomic duckweed plant produced according to the method of claim 40.

42. A method for obtaining a duckweed plant containing stably transformed plastids, said method comprising the steps of:
a) providing a nucleic acid molecule of claim 14, wherein said nucleic acid molecule comprises a marker sequence conferring a selectable phenotype to duckweed cells;
b) bombarding a duckweed nodule with said nucleic acid molecule absorbed to a microprojectile under conditions such that the nucleotide sequence heterologous to a duckweed plastome is introduced into at least one plastid of said duckweed nodule;
c) maintaining the duckweed nodule in a selection medium which permits the survival of duckweed cells having the selectable phenotype to thereby select for duckweed cells containing the marker sequence; and
d) regenerating a duckweed plant from said duckweed nodule.

43. The method of claim 42, wherein the selection medium preferentially permits survival of duckweed cells having substantially all plastids transformed with the nucleic acid molecule.

44. A transplastomic duckweed plant produced according to the method of claim 42.

45. A method for producing a polypeptide of interest, said method comprising
a) bombarding a duckweed nodule with a nucleic acid molecule of claim 14 absorbed to a microprojectile under conditions such that said nucleotide sequence heterologous to a duckweed plastome is introduced into at least one plastid of said duckweed nodule; and
b) culturing said duckweed plant under conditions such that the polypeptide of interest is expressed from said nucleic acid molecule.

46. A method for producing a polypeptide of interest, said method comprising
a) bombarding a duckweed nodule with a nucleic acid molecule of claim 14 absorbed to a microprojectile under conditions such that said nucleotide sequence heterologous to a duckweed plastome is introduced into at least one plastid of said duckweed nodule;
b) regenerating a duckweed plant from said duckweed nodule; and
c) culturing said duckweed plant under conditions such that the polypeptide of interest is expressed from said nucleic acid molecule.

47. A duckweed cell having plastids that are stably transformed with at least one nucleotide sequence heterologous to a duckweed plastome, wherein the nucleotide sequence heterologous to a duckweed plastome is operably linked to an expression control sequence that is capable of functioning in the plastid.

48. A method for producing one or more polypeptides of interest, said method comprising:
a) providing a duckweed cell according to claim 27, wherein said nucleotide sequence heterologous to a duckweed plastome encodes one or more polypeptides of interest; and
b) culturing said duckweed cell under conditions such that the polypeptide of interest is expressed.

49. A nucleic acid molecule having a nucleotide sequence selected from the group consisting of:
a) the nucleotide sequence set forth in SEQ ID NO:3;
b) the nucleotide sequence of a fragment of the nucleotide sequence set forth in SEQ ID NO:3, wherein said fragment comprises at least 100 contiguous nucleotides of the nucleotide sequence set forth in SEQ ID NO:3;
c) the nucleotide sequence of a fragment of the nucleotide sequence set forth in SEQ ID NO:3, wherein said fragment comprises at least 200 contiguous nucleotides of the nucleotide sequence set forth in SEQ ID NO:3;
d) the nucleotide sequence of a fragment of the nucleotide sequence set forth in SEQ ID NO:3, wherein said fragment comprises at least 400 contiguous nucleotides of the nucleotide sequence set forth in SEQ ID NO:3;
e) the nucleotide sequence of a fragment of the nucleotide sequence set forth in SEQ ID NO:3, wherein said fragment comprises at least 600 contiguous nucleotides of the nucleotide sequence set forth in SEQ ID NO:3;
f) the nucleotide sequence of a fragment of the nucleotide sequence set forth in SEQ ID NO:3, wherein said fragment comprises at least 800 contiguous nucleotides of the nucleotide sequence set forth in SEQ ID NO:3;
g) the nucleotide sequence of a fragment of the nucleotide sequence set forth in SEQ ID NO:3, wherein said fragment comprises at least 1000 contiguous nucleotides of the nucleotide sequence set forth in SEQ ID NO:3;
h) a nucleotide sequence having at least 90% sequence identity with the nucleotide sequence set forth in SEQ ID NO:3, wherein said nucleotide sequence having at least 99% sequence identity with SEQ ID NO:3 is capable of homologous recombination with a duckweed chloroplast genome; and
i) a nucleotide sequence having at least 95% sequence identity with the nucleotide sequence set forth in SEQ ID NO:3, wherein said nucleotide sequence having at least 95% sequence identity with SEQ ID NO:3 is capable of homologous recombination with a duckweed chloroplast genome.
